(19) United States
(12) Patent Application Publication
(10) Pub. No.: US 2024/0115428 A1
(43) Pub. Date: Apr. 11, 2024

(54) Period Pad Device

(71) Applicant: Liliana Serrano, Ruskin, FL (US)

(72) Inventor: Liliana Serrano, Ruskin, FL (US)

(21) Appl. No.: 18/479,891

(22) Filed: Oct. 3, 2023

Related U.S. Application Data
(60) Provisional application No. 63/414,934, filed on Oct. 11, 2022.

(51) Int. Cl. A61F 13/472; A61F 13/505; A61F 13/72; A61F 13/74

(52) U.S. Cl. CPC A61F 13/472; A61F 13/505; A61F 13/72; A61F 13/74

(57) ABSTRACT

A period pad device is enclosed. The device is a set of modified undergarments for women with an integrated sanitary pad. The period pad device offers protection against bodily fluid leakage during the menstrual cycle. The period pad device comprises a body component with a pad sewn into the base of underwear. The pad that is sewn into the body component is positioned over the vaginal opening to absorb fluids. The body component can be thrown away after use or washed and reused. Thus, the period pad device eliminates the worry of accidentally staining standard undergarments during a period. The device can be available in several colors, styles, sizes, and designs with various size, measurement, construction, and design specifications. The device can also comprise a taller compression fit in addition to the body component.

### CROSS-REFERENCE TO RELATED APPLICATION

The present application claims priority to, and the benefit of, U.S. Provisional Application No. 63/414,934, which was filed on Oct. 11, 2022, and is incorporated herein by reference in its entirety.

### FIELD OF THE INVENTION

The present invention relates generally to the field of period pad devices. More specifically, the present invention relates to a set of modified undergarments for women with an integrated sanitary pad. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, this invention relates to improvements in period pad devices. Generally, women must wear sanitary pads or tampons to prevent blood from staining the undergarments during a monthly menstrual cycle. Despite forming a barrier between the fabric and bodily fluids, a pad or tampon can sometimes leak and lead to stains. Accordingly, people may be looking for a less embarrassing way to prevent bodily fluids on their undergarments.

Further, the use of pads or tampons to protect undergarments from bodily fluids is well known in the prior art. However, it would be desirable to include a pad within the undergarments for the purpose of preventing staining on the undergarments or other clothing materials. For example, the protective menstrual undergarment would provide added comfort and increased protection against leakage for menstruating women. By eliminating stains, embarrassment will be avoided. In addition, the undergarment will give a woman confidence, reassurance, and peace of mind that her underwear and outerwear would always be fresh and clean. The undergarment can be disposable or washable and incorporates a number of features to secure a pad in place. The period undergarment will also feature a body portion made from a stretchable fabric, which will flatten a bloated stomach. The present invention is versatile, in that it can be used by women or even men experiencing any type of discharge, such as the elderly who have bladder problems, men or women during hospital stays, and women with yeast infections or bladder infections.

Accordingly, there is a demand for an improved period pad device that can protect against leakage from conventional pads and tampons. More particularly, there is a demand for a completely leakproof period undergarment that can protect women during their monthly menstrual cycles.

Therefore, there exists a long-felt need in the art for a period pad device that provides users with a combination undergarment and sanitary pad capable of absorbing bodily fluids during the menstrual cycle. There is also a long-felt need in the art for a period pad device that allows women to wear a combination undergarment rather than separate underwear and a pad or tampon. Further, there is a long-felt need in the art for a period pad device that improves protection against bodily fluid stains, eliminating the worry of permanently staining standard undergarments. Moreover, there is a long-felt need in the art for a device that enables users to simply throw away the undergarments after a single use to replace them with a new pair with a clean pad. Further, there is a long-felt need in the art for a period pad device that enables users to wash the undergarments and wear them multiple times instead of replacing them after each use. Finally, there is a long-felt need in the art for a period pad device that brings comfort and security to women during their menstrual cycles.

The subject matter disclosed and claimed herein, in one embodiment thereof, comprises a period pad device. The device is a set of modified undergarments for women with an integrated sanitary pad. The period pad device offers protection against bodily fluids during the menstrual cycle. The period pad device comprises a body component, wherein the body component comprises a fabric component with a front and back side sewn together and an elastic waistband and also comprises a pad component sewn into the base of the fabric component. The pad component that is sewn into the fabric component is positioned over the vaginal opening to absorb fluids. The body component can be thrown away after use or washed and reused. Thus, the period pad device eliminates the worry of accidentally staining standard undergarments during a period. The device can be available in several colors, styles, sizes, and designs with various sizes, measurements, construction, and design specifications. The device can also comprise a taller compression fit in addition to the body component.

In this manner, the period pad device of the present invention accomplishes all the foregoing objectives and provides users with a device that provides a combination undergarment and sanitary pad capable of absorbing bodily fluids. The device is a modified undergarment that has a sanitary pad integrated into the lining of it. The device can be manufactured of any suitable material known in the art.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a period pad device. The device is a set of modified undergarments for women with an integrated sanitary pad. The period pad device comprises a body component that is configured in a conventional underwear shape. The body component would comprise a pad component that is sewn into the bottom lining of the fabric component and positioned over the vaginal opening. Thus, each period pad device functions as a way to absorb bodily fluids during a menstrual cycle without the worry of permanently staining standard undergarments or outerwear.

In one embodiment, the period pad device comprises a fabric component that is worn around the lower torso of a woman, much like conventional underwear. The body component is made up of the fabric component and has a pad component sewn into the crotch position of the fabric component so that the pad component covers the vaginal opening. The sewn-in pad component is then covered by the material the fabric component is made of. The material of the body component can be any suitable sanitary material known in the art. The body component can be composed of several colors, styles, sizes, and designs.

In one embodiment, the period pad device comprises a body component that is disposable. Once used, the user may throw the entire body component away and use a new one. Further, the pad component should be securely sewn into the body component and should also be made of suitable disposable materials.

In one embodiment, the body component is made from washable materials along with the pad component sewn into the body component. Further, after use, the body component, along with the sewn-in pad component, can be washed in a traditional washing machine for reuse. In another embodiment, the fabric component is made of a washable material while the pad component is made from disposable material. Further, the pad would not be sewn into the body component but rather fastened inside with additional fasteners, such as hook and loop fasteners or any other suitable fastener known in the art. The pad component can then be removed from the body component once used and disposed of while the fabric component can be washed. A new pad component can then be placed and fastened into the fabric component.

In one embodiment, the body component comprises two elastic bands which are placed near the front and the back of the crotch position. These bands would ensure the pad component is staying centered to the crotch position and will not move and cause leakage during use.

In one embodiment, the period pad device comprises a compressible component that is positioned above the body component. This compressible component is made of a compressible material that can extend from the body component until it completely encompasses the user's stomach. Further, the compressible material would be able to compress a woman's stomach which might be bloated during her menstrual cycle.

In one embodiment, the compressible component of the body component contains a small pocket on the front side. This small pocket is made to be within the body component and is small enough to not be seen while the body component is in use, and yet large enough so that a portable heating pad may be inserted into the small pocket. This pocket allows the heating pad to warm the uterus portion of the skin to combat cramps during a menstrual cycle.

In one embodiment, the period pad device can be used for more than just women during their menstrual cycles. The body component can adapt to fit men's underwear for purposes such as hospital stays, bladder infections, and elderly loss of bladder control as well. It can also be used for those purposes in women as well as with a yeast infection or other discharge.

In use, the period pad device consists of a conventionally shaped pair of underwear with an integrated sanitary pad. The sanitary pad is properly positioned and sewn into the body component. The period pad device can be washable or disposable in order to maintain freshness. Thus, bodily fluids are not leaked from the body component.

In one embodiment, the period pad device can be utilized with any suitable underwear of various colors, sizes, styles, and designs. The material may be any washable or disposable material known in the art that provides comfort and security for the user. The sanitary pad may also be made of any suitable washable or disposable material known in the art. The fasteners for the removable pad may also be any secure fastener known in the art that provides comfort and security for the user.

In yet another embodiment, the period pad device comprises a plurality of indicia.

In yet another embodiment, a method of protecting regular undergarments and outerwear from bodily fluid stains is disclosed. The method includes the steps of providing a combination undergarment and sanitary pad device capable of absorbing bodily fluids during the menstrual cycle. The method also comprises wearing the device during the menstrual cycle as a conventional undergarment. Further, the method comprises securing the device to a user via stepping into the leg openings of the body component. Further, the method comprises pulling up the body component to position the pad component in place. Finally, the method comprises taking off the device and disposing of it in the proper form.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view and the rear perspective view of one embodiment of the period pad device of the present invention showing the device with the sanitary pad integrated into different styles of underwear;

FIG. 2 illustrates a front perspective view of different styles of one embodiment of the period pad device of the present invention showing how the body component varies per requested style;

FIG. 3 illustrates a perspective view of one embodiment of the period pad device on a user showing how the body component should be worn and how it should be discarded after every use to maintain sanitary conditions; and

FIG. 4 illustrates a flowchart showing the method of wearing the combination undergarment in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a period pad device that provides users with a combination undergarment and sanitary pad capable of absorbing bodily fluids during the menstrual cycle. There is also a long-felt need in the art for a period pad device that allows women to wear the combination undergarment rather than separate underwear and a pad or tampon. Further, there is a long-felt need in the art for a period pad device that improves protection against bodily fluid stains. Moreover, there is a long-felt need in the art for a device that eliminates the worry of permanently staining standard undergarments. Further, there is a long-felt need in the art for a period pad device that enables users to simply throw away the undergarments after a single use to replace it with a new pair with a clean pad or enables users to wash the undergarment after a single use to maintain sanitary conditions and reuse the undergarment. Finally, there is a long-felt need in the art for a period pad device that can be used as men's underwear for different discharge situations as well.

The present invention, in one exemplary embodiment, is a novel period pad device. The device is a set of modified undergarments for women with an integrated sanitary pad. The period pad device comprises a body component that is worn around the lower torso of a woman. The body component comprises a fabric component that comprises a front and back side that is secured around the crotch position so as to form two openings for the insertion of a pair of legs. The fabric component is fabricated out of a soft stretch material and an elastic waistband is attached to an open top of the fabric component to keep the open top end in a snug fit around the waist. A pad component is then sewn or fastened into the crotch position of the fabric component. The body component would have a piece of fabric covering the pad so that it is not seen from the outside. In one embodiment, the body component can also comprise a tall compression component with a small pocket for help compressing bloating stomachs and holding a heating pad. Thus, each period pad device functions as a suitable way of protecting users from stains from bodily fluids on their regular undergarments or outerwear. The present invention also includes a novel method of protecting regular undergarments and outerwear from bodily fluid stains. The method includes the steps of providing a combination undergarment and sanitary pad capable of absorbing bodily fluids during the menstrual cycle. The method also comprises wearing the device during the menstrual cycle as a conventional undergarment. Further, the method comprises securing the device to a user via stepping into the leg openings of the body component. Further, the method comprises pulling up the body component to position the pad component in place. Finally, the method comprises taking off the device and disposing of it in the proper form.

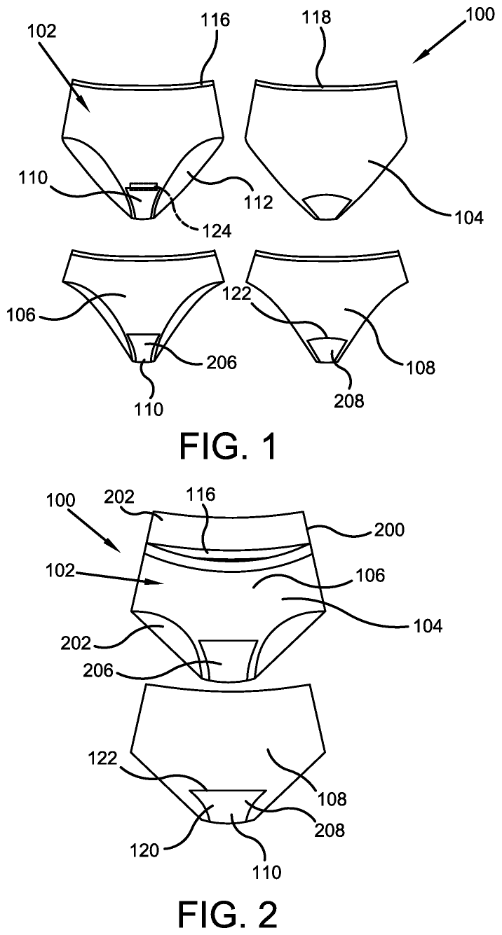

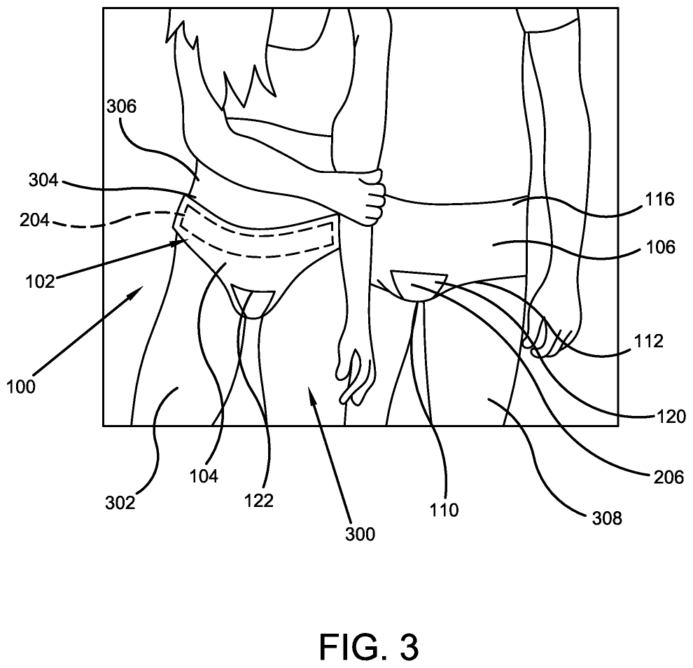

Referring initially to the drawings, FIGS. 1-2 illustrate a perspective view of one embodiment of the period pad device 100 of the present invention. In the present embodiment, the period pad device 100 comprises a body component 102 which comprises a fabric component 104 of a front side 106 and back side 108 that is secured around the crotch position 110 so as to form two openings 112 for insertion of a pair of legs (as seen in FIG. 3, 302). The fabric component 104 is fabricated out of a soft stretch material and an elastic waistband 116 is attached about an open top 118 of the fabric component 104 to keep the open top 118 end in a snug fit around a user's waist (as seen in FIG. 3, 304). The body component 102 also comprises a pad component 120 which is positioned at the crotch position 110 of the fabric component. The pad component 120 can either be sewn or removably fastened inside the fabric component 104.

Furthermore, the pad component 120 is integrated into the fabric component 104, such that the pad component 120 is covered by the same material the rest of the body component 102 is made of. The material of the body component 102 can be any suitable sanitary material known in the art. Further, the body component 102 can be composed of several colors, styles, sizes, and designs.

As shown in FIG. 2, when the pad component 120 is removably fastened into the crotch position 110, it can be secured in place by two elastic bands 122 that sit at either end of the crotch position 110 to ensure the pad component 120 does not move during use. Further, additional fasteners 124 allow the pad component 120 to be replaced and resecured as needed. These additional fasteners 124 can be Velcro, adhesives, etc., or any other suitable removable fasteners as is known in the art.

When using the two elastic bands 122, the bands 122 would be placed near the front 206 and the back 208 of the crotch position 110. These elastic bands 122 would stabilize the pad component 120 by pinning the pad component 120 into the fabric component 104 until the additional fasteners 124 are released. This would ensure that the pad component 120 is staying center to the crotch position 110 and will not move and cause leakage during use.

Generally, the period pad device 100 can be utilized with any fabric component 104 of various colors, sizes, styles, and designs. The material may be any washable or disposable material known in the art that provides comfort and security for the user (as seen in FIG. 3, 300). The pad component 106 may also be made of any suitable material washable or disposable known in the art. The additional fasteners 124 for the pad component 120 may also be any secure fastener 124 known in the art that provides comfort and security for the user 300.

Furthermore, in one embodiment, the period pad device 100 comprises a compressible component 200 integrated above the body component 102 and made from a compressible material. This compressible component 200 can extend from the body component 102 until it completely encompasses the user's stomach (as seen in FIG. 3, 306). Further, the compressible component 200 would be able to compress a user's stomach 306 which might be bloated during a menstrual cycle.

In one embodiment, the compressible component 200 contains a small pocket 202 on the front side 106 of the body component. This small pocket 202 is made to be within the compressible component 200 and is small enough to not be seen while the body component 102 is in use, yet large enough so that a portable heating pad 204 or other suitable heating components may be inserted into the small pocket 202. This small pocket 202 allows the portable heating pad 204 to warm the user's skin (as seen in FIG. 3, 308) to combat cramps during a menstrual cycle.

As shown in FIG. 3, the period pad device 100 comprises a body component 102 that is disposable. Once used, the user 300 may throw the entire body component 102 away and use a new one. Further, in this embodiment, the pad component 120 should be securely sewn into the body component 102 and should also be made of disposable materials.

In another embodiment, the body component 102 is made from washable materials along with the pad component 120 when it is sewn into the body component 102. Further, after use, the body component 102 can be washed in a traditional washing machine for reuse. In another embodiment, the body component 102 is made of a washable material while the pad component 120 is made from disposable material. Further, in this embodiment, the pad component 120 would not be sewn into the body component 102 but rather, fastened inside with additional fasteners 124 such as hook and loop fasteners or any other suitable fastener known in the art. The pad component 120 can then be removed from the body component 102 once used and disposed of while the body component 102 can be washed. A new pad component 120 can then be placed and fastened into the body component 102.

Generally, the period pad device 100 can also be used for more than just women during their menstrual cycles. The body component 102 can adapt to fit men's underwear for purposes such as hospital stays, bladder infections, and elderly loss of bladder control as well. It can also be used for those purposes in women as well, along with in the case of a yeast infection or other discharge.

Certain terms are used throughout the following description and claim to refer to particular features or components. As one skilled in the art will appreciate, different users, may refer to the same feature or component by different names. This document does not intend to distinguish between components or features that differ in name but not structure or function. As used herein “period pad device”, “pad device”, “undergarment device”, and “device” are interchangeable and refer to the period pad device 100 of the present invention.

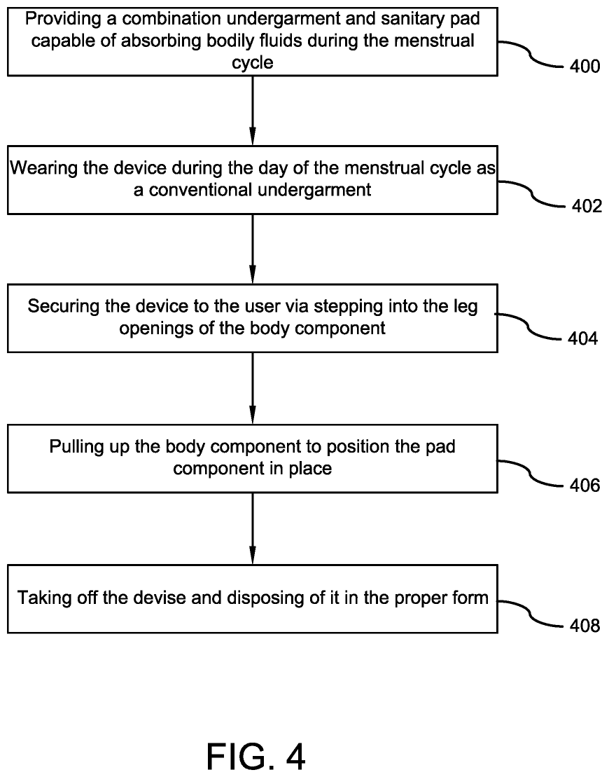

Notwithstanding the foregoing, the period pad device 100 of the present invention can be of any suitable size and configuration as is known in the art without affecting the overall concept of the invention, provided that it accomplishes the above-stated objectives. One of ordinary skill in the art will appreciate the period pad device 100 as shown in FIGS. 1-4 are for illustrative purposes only, and many other sizes and shapes of the period pad device 100 are well within the scope of the present disclosure. Although the dimensions of the period pad device 100 are important design parameters for user convenience, the period pad device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A period pad device that provides a user with a modified undergarment with an integrated pad for protecting against bodily fluid stains, the period pad device comprising:
a body component comprising a fabric component and a pad component;
wherein the pad component is secured to a crotch position of the fabric component; and
further wherein the body component is worn as a conventional undergarment and prevents bodily fluid from staining clothing.

2. The period pad device of claim 1, wherein the pad component is sewn into the crotch position of the fabric component.

3. The period pad device of claim 2, wherein the body component is composed of disposable material and thrown away after use.

4. The period pad device of claim 2, wherein the body component is composed of washable material and washed clean after each use.

5. The period pad device of claim 1, wherein the pad component is removably fastened inside of the fabric component.

6. The period pad device of claim 5, wherein the pad component is made of disposable material while the fabric component is made from washable material.

7. The period pad device of claim 5, wherein the pad component is fastened into the body component with hook and loop fasteners.

8. The period pad device of claim 1, wherein the body component comprises a compressible component that is positioned above a waistband of the body component and is made of compressible material and fully wraps around a user's stomach.

9. The period pad device of claim 8, wherein the compressible component comprises a small pocket, which can hold a portable heating pad.

10. The period pad device of claim 1, wherein the pad component, when removable, is surrounded by elastic bands at the front and back of the crotch position of the body component which keep the pad component in place.

11. The period pad device of claim 1, wherein the body component comprises a front side, a back side secured together at the crotch position, with two leg openings, and an elastic waistband.

12. The period pad device of claim 1 further comprising a plurality of indicia.

13. A period pad device that provides a user with a modified undergarment with an integrated pad for protecting against bodily fluid stains, the period pad device comprising:
a body component comprising a fabric component and a pad component which is integrated into the fabric component; and
wherein the body component comprises a front side, a back side secured together at the crotch position, with two leg openings, and an elastic waistband;
wherein the body component is shaped and worn in the fashion of conventional undergarments;
wherein the pad component is integrated into the fabric component, such that the pad component is sewn into the crotch position and absorbs bodily fluids that are excreted; and
further wherein the body component is composed of disposable material and thrown away after use.

14. The period pad device of claim 13, wherein the body component is manufactured of a washable material.

15. The period pad device of claim 13, wherein the fabric component is manufactured of washable material and the pad component is removable and manufactured of disposable material.

16. The period pad device of claim 15, wherein the pad component is removably fastened inside of the fabric component via Velcro.

17. The period pad device of claim 16, wherein the pad component, when removable, is secured in place during use by a pair of elastic bands on either end of a crotch position.

18. The period pad device of claim 13, wherein the body component comprises a compressible component that is positioned above a waistband of the body component and is made of compressible material and fully wraps around a user's stomach.

19. The period pad device of claim 13, wherein the compressible component comprises a small pocket, which can hold a portable heating pad.

20. A method of protecting regular undergarments and outerwear from bodily fluid stains, the method comprising the following steps:
providing a combination undergarment and sanitary pad capable of absorbing bodily fluids during the menstrual cycle;
wearing the device during the day of the menstrual cycle as a conventional undergarment; securing the device to a user via stepping into the leg openings of the body component; pulling up the body component to position the pad component in place; and
taking off the device and disposing of it in the proper form.
